(19) United States
(12) Patent Application Publication
(10) Pub. No.: US 2025/0292457 A1
(43) Pub. Date: Sep. 18, 2025

(54) SYSTEMS AND METHODS FOR ATTENUATION CORRECTION

(71) Applicant: SHANGHAI UNITED IMAGING HEALTHCARE CO., LTD., Shanghai (CN)

(72) Inventors: Tiantian LI, Houston, TX (US); Liuchun HE, Shanghai (CN); Zhongzhi LIU, Wuhan (CN); Songsong TANG, Shanghai (CN); Yun DONG, Shanghai (CN); Hongdi LI, Houston, TX (US)

(73) Assignee: SHANGHAI UNITED IMAGING HEALTHCARE CO., LTD., Shanghai (CN)

(21) Appl. No.: 19/070,499

(22) Filed: Mar. 4, 2025

(30) Foreign Application Priority Data
Mar. 13, 2024 (CN) 202410288352.5

(51) Int. Cl. G06T 11/00

(52) U.S. Cl. CPC G06T 11/005; G06T 11/006; G06T 2210/41; G06T 2211/424; G06T 2211/452

(57) ABSTRACT

Embodiments of the present disclosure provide a method, a system, and a medium for attenuation correction. The method includes obtaining radiological coincidence event data of a target object using an imaging device; obtaining transmission data related to the target object; and obtaining an attenuation-corrected radiological image by performing an attenuation correction and reconstruction based on the transmission data and the radiological coincidence event data.

### CROSS-REFERENCE TO RELATED APPLICATION

This application claims priority of Chinese Patent Application No. 202410288352.5, filed on Mar. 13, 2024, the contents of which are entirely incorporated herein by reference.

### TECHNICAL FIELD

The present disclosure relates to the technical field of positron emission tomography imaging, and in particular to methods, systems, and storage media for attenuation correction.

### BACKGROUND

Attenuation correction is important for positron emission tomography (PET). Attenuation correction is usually achieved by fusing PET data with computed tomography (CT) data. The CT data provides density information of a tissue, which is used to calculate attenuation factors of photons. These attenuation factors are then applied to the PET data to remove an attenuation effect of the photons in the tissue. However, an attenuation map (μ-map) derived based on the CT data has some limitations (e.g., a high radiation dose, a metal artifact, a beam-hardening artifact, a truncation of a great patient, and a patient motion between PET/CT scan may affect the derivation of the attenuation map).

There is therefore a need for systems and methods for attenuation correction that provide an accurate attenuation correction result.

### SUMMARY

One or more embodiments of the present disclosure provide a method for attenuation correction. The method includes: obtaining radiological coincidence event data of radiation of a target object using an imaging device; obtaining transmission data related to the target object; and obtaining an attenuation-corrected radiological image by performing an attenuation correction and reconstruction based on the transmission data and the radiological coincidence event data. In some embodiments, the transmission data includes at least one of: backscattering coincidence event data of the target object or lutetium background event data of the imaging device.

One or more embodiments of the present disclosure provide a system for attenuation correction for implementing the method for attenuation correction. The system includes: a first obtaining module configured to obtain radiological coincidence event data of radiation of a target object using an imaging device; a second obtaining module configured to obtain transmission data related to the target object; and a reconstruction module configured to obtain an attenuation-corrected radiological image by performing an attenuation correction and reconstruction based on the transmission data and the radiological coincidence event data.

One or more embodiments of the present disclosure provide a non-transitory computer-readable storage medium storing computer instructions. When reading the computer instructions in the storage medium, a computer implements the method for attenuation correction.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an application scenario of a system for attenuation correction according to some embodiments of the present disclosure;

FIG. 2 is a block diagram illustrating an exemplary system for attenuation correction according to some embodiments of the present disclosure;

FIG. 3 is a flowchart illustrating an exemplary process for attenuation correction according to some embodiments of the present disclosure;

FIG. 4 is a schematic diagram illustrating an exemplary backscattering event according to some embodiments of the present disclosure;

FIG. 5 is a schematic diagram illustrating an exemplary lutetium background event according to some embodiments of the present disclosure;

FIG. 6 is a flowchart illustrating an exemplary process for obtaining an attenuation-corrected radiological image of a target object according to some embodiments of the present disclosure;

FIG. 7 is a schematic diagram illustrating an exemplary process for obtaining an attenuation-corrected radiological image by performing an iterative reconstruction based on backscattering coincidence event data of the target object according to some embodiments of the present disclosure;

FIG. 8 is a schematic diagram illustrating an exemplary process for obtaining an attenuation-corrected radiological image by performing an iterative reconstruction based on lutetium background event data according to some embodiments of the present disclosure;

FIG. 9 is a schematic diagram illustrating an exemplary process for obtaining an attenuation-corrected radiological image by performing an iterative reconstruction based on lutetium background event data and backscattering coincidence event data according to some embodiments of the present disclosure;

FIG. 10 is a schematic diagram illustrating an exemplary symmetry of a PET system according to some embodiments of the present disclosure; and

FIG. 11 is a schematic diagram illustrating exemplary attenuation images according to some embodiments of the present disclosure.

### DETAILED DESCRIPTION

To more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system,” “device,” “unit” and/or “module” as used herein is a way to distinguish between different components, elements, parts, sections or assemblies at different levels. However, the words may be replaced by other expressions if other words accomplish the same purpose.

As shown in the present disclosure and in the claims, unless the context clearly suggests an exception, the words “a,” “one,” “an” and/or “the” do not refer specifically to the singular, but may also include the plural. Generally, the terms “including” and “comprising” suggest only the inclusion of clearly identified operations and elements that do not constitute an exclusive list, and the method or device may also include other operations or elements.

Flowcharts are used in the present disclosure to illustrate operations performed by a system in accordance with embodiments of the present disclosure. It should be appreciated that the preceding or following operations are not necessarily performed in an exact sequence. Instead, the operations can be processed in reverse order or simultaneously. Also, it is possible to add other operations to these processes or remove an operation or operations from them.

An attenuation correction (AC) is an important operation in positron emission tomography (PET) imaging. During PET imaging, emitted positrons interact with surrounding tissues and generate 511 keV photons, which are subject to different degrees of attenuation as the photons pass through the tissues of a body. Therefore, correction on these attenuations is required to obtain a more accurate PET image.

In PET/computed tomography (CT) imaging, the attenuation correction is usually achieved by fusing PET data with CT data. The CT data provides information about density of the tissue, which is used to calculate attenuation factors of the photons. These attenuation factors are then applied to the PET data to remove an attenuation effect of the photons in the tissue.

However, an attenuation map (μ-map) derived based on the CT data has some limitations. For example, a high radiation dose, a metal artifact, a beam-hardening artifact, a truncation of a great patient, and a patient motion between PET/CT scan may affect the derivation of the attenuation map.

The prior art performs the attenuation correction by jointly reconstructing an attenuation map and an activity map, and this allows for a direct extraction of attenuation factor information directly from radiological data. A maximum likelihood estimation (MLAA) of the attenuation map and the activity map is first calculated, and the PET and an attenuation image reconstruction are iterated alternatively in sequence using a maximum likelihood expectation maximization (MLEM) and a transmission tomography maximum likelihood (MLTR) algorithm. Similar to the MLAA, some studies propose the maximum likelihood estimation of the activity map and the attenuation correction factor (MLACF), where the attenuation factor is estimated after each update of the activity image or at each update of the activity image.

A potential problem with such algorithms is that, on the one hand, the MLAA or the MLACF may converge to a local optimum solution if initial values are not good enough. On the other hand, the MLAA and the MLACF use the same set of radiological data for estimating the activity map and the attenuation map, a crosstalk problem may also affect the speed of convergence and the quality of the estimated images.

To address the above issues, external rotated sources are used in some studies to reconstruct the attenuation map used directly for the attenuation correction or as the initial values of the attenuation map in the MLAA. A disadvantage of external source scanning is that an additional device is required, which increases the complexity of system design and user operation while also increasing a radiation dose received by a patient.

In some embodiments of the present disclosure, a method for attenuation correction is provided. In the method, an attenuation-corrected radiological image of a target object is reconstructed by obtaining transmission data, and by obtaining radiological coincidence event data of the target object. The transmission data includes backscattering coincidence event data of the target object. Without an aid of the CT data, the attenuation image or the attenuation factors are estimated and corrected without increasing the complexity of the system, a scanning time, or the radiation dose to the patient.

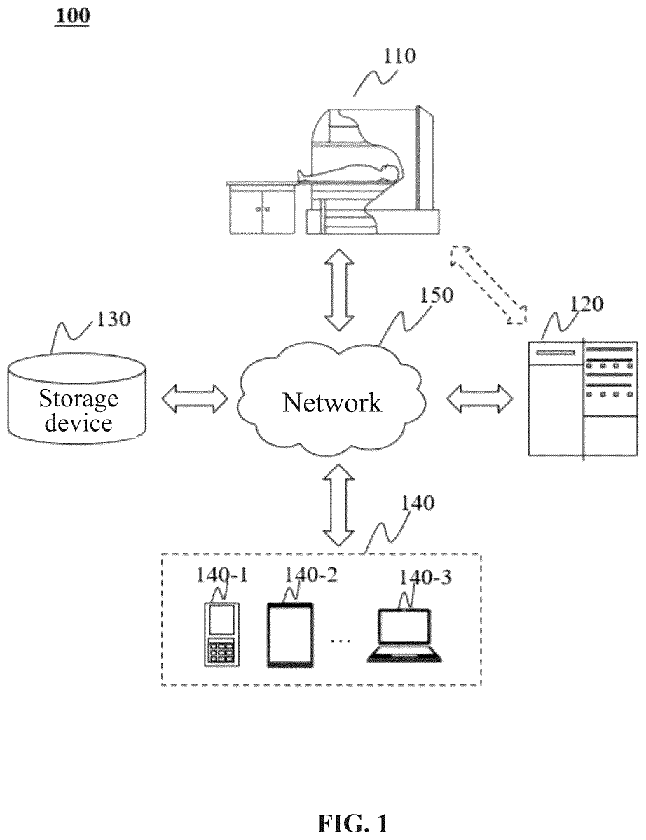

FIG. 1 is a schematic diagram illustrating an application scenario of a system for attenuation correction according to some embodiments of the present disclosure.

As shown in FIG. 1, in some embodiments, an application scenario of the system 100 for attenuation correction includes a scanning device 110, a processing device 120, a storage device 130, a terminal 140, and/or a network 150.

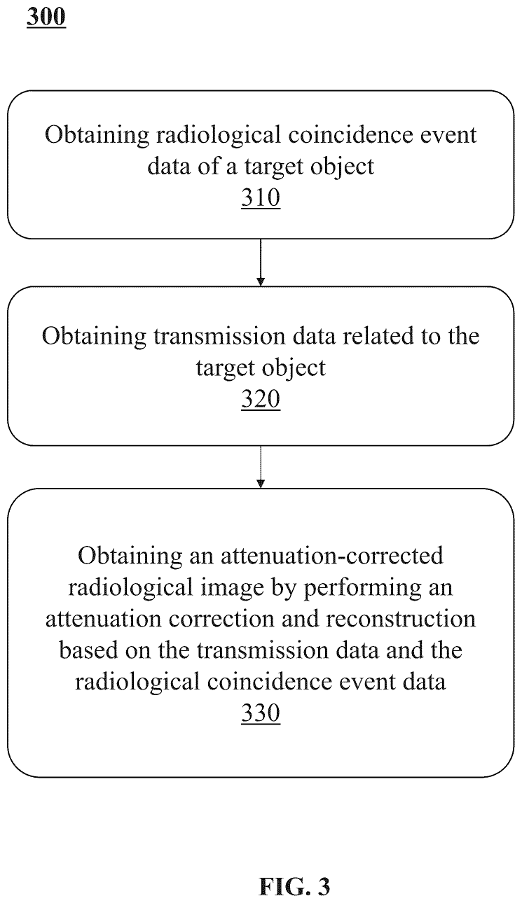

The scanning device 110 refers to a medical device that reproduces an internal structure of an object (e.g., a human body) as an image. In some embodiments, the scanning device 110 can be any medical device that images or treats a designated part of an object by means of radionuclides, for example, a PET device, a CT device, a PET-CT device, etc. The scanning device 110 provided above is for illustrative purposes only and is not a limitation of the scope of the present disclosure. A detector in the scanning device 110 may receive radiation from a radiation source and meter a received radiation. The detector includes a plurality of detector units arranged in one or more rings. In some embodiments, the scanning device 110 sends data and information related to the detector, such as an energy value of radiated photons received by the detector, an output value of the detector, etc., to the processing device 120. In some embodiments, the scanning device 110 collects transmission data, radiological coincidence event data of the scanning target object, etc., and sends them to the processing device 120. More descriptions about the transmission data, the target object, and the radiological coincidence event data may be found in FIG. 3 and the related descriptions. In some embodiments, the scanning device 110 receives an instruction, etc., sent by a physician through the terminal 140, and performs relevant operations, e.g., a radiation imaging, etc., according to the instruction. In some embodiments, the scanning device 110 exchanges data and/or information with other components of the system 100 (e.g., the processing device 120, the storage device 130, the terminal 140) through the network 150. In some embodiments, the scanning device 110 is directly connected to other components in the application scenario 100 of the system for attenuation correction. In some embodiments, the one or more components of the application scenario 100 of the system for attenuation correction (e.g., the processing device 120, the storage device 130) are included within the scanning device 110.

The processing device 120 may process the data and/or information obtained from other devices or components of the system, and based on the data, the information, and/or a processing result, the processing device 120 may perform operations for correcting a scanning image as illustrated in some embodiments of the present disclosure, so as to accomplish one or more functions described in some embodiments of the present disclosure. For example, the processing device 120 obtains an attenuation-corrected radiological image by performing an attenuation correction and reconstruction based on the transmission data and the radiological coincidence event data. In some embodiments, the processing device 120 obtains pre-stored data and/or information, e.g., the transmittance data, the radiological coincidence event data, various calculation formulas, etc., from the storage device 130 for performing the method for attenuation correction according to some embodiments of the present disclosure.

In some embodiments, the processing device 120 includes one or more sub-processing devices (e.g., a single-core processing device or a multi-core processing device). Merely by way of example, the processing device 120 includes a central processing unit (CPU), an application-specific integrated circuit (ASIC), a graphics processor (GPU), a digital signal processor (DSP), a field programmable gate array (FPGA), a microcontroller unit (MCU), a reduced instruction set computer (RISC), a microprocessor, etc., or any combination of the above.

The storage device 130 stores data or information generated by other devices. In some embodiments, the storage device 130 stores the data and/or information collected by the scanning device 110, for example, the transmission data, the radiological coincidence event data, etc. The storage device 130 may include one or more storage components, each of which is an independent device or a part of other devices. The storage device may be local or be implemented through a cloud. In some embodiments, one or more components of the system 100 (e.g., the scanning device 110, the processing device 120, the terminal 140) include their own storage components.

The terminal 140 may control an operation of the scanning device 110. A physician may issue an operational instruction to the scanning device 110 through the terminal 140 to enable the scanning device 110 to complete a specified operation, such as irradiating a specified body part of the patient for imaging. In some embodiments, the terminal 140 is instructed to enable the processing device 120 to perform the method for attenuation correction according to some embodiments of the present disclosure. In some embodiments, the terminal 140 receives the attenuation-corrected radiological image, etc., from the processing device 120, so that the physician accurately determines a situation of the patient for an effective and targeted examination and/or treatment. In some embodiments, the terminal 140 is a mobile device 140-1, a tablet computer 140-2, a laptop computer 140-3, a desktop computer, and other input and/or output devices, or any combination thereof.

The network 150 may connect components of the system and/or connect the system to external resource portions. The network 150 may enable communications between components and other portions outside the system, so as to facilitate the exchange of the data and/or information. In some embodiments, the one or more components of the system 100 (e.g., the scanning device 110, the processing device 120, the storage device 130, the terminal 140) send the data and/or information to other components through the network 150. In some embodiments, the network 150 is any one or more of a wired network or a wireless network.

It should be noted that the foregoing description is provided for illustrative purposes only and is not intended to limit the scope of the present disclosure. For those skilled in the art, a wide variety of changes and modifications may be made under the guidance of the contents of the present disclosure. Features, structures, methods, and other characteristics of the exemplary embodiments described herein may be combined in various ways to obtain additional and/or alternative exemplary embodiments. For example, the processing device 120 is based on a cloud calculation platform, such as a public cloud, a private cloud, a community, and a hybrid cloud, etc. However, these changes and modifications do not depart from the scope of the present disclosure.

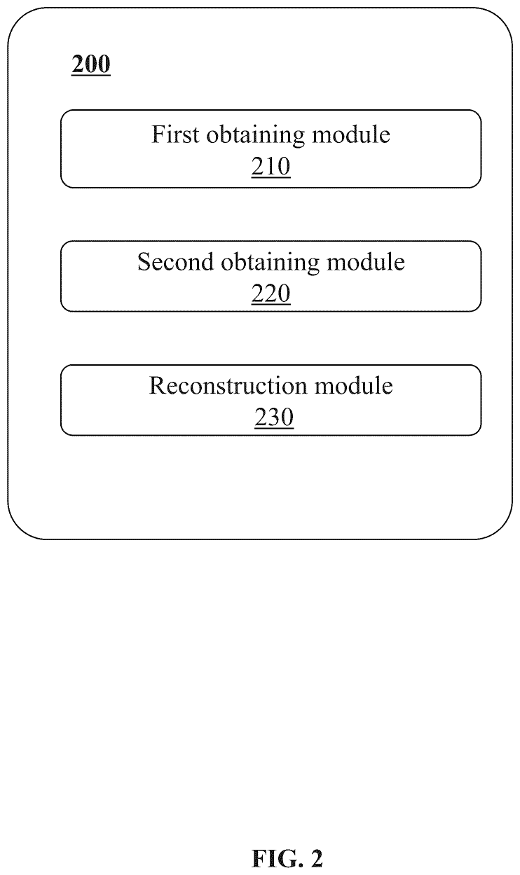

FIG. 2 is a block diagram illustrating an exemplary system for attenuation correction according to some embodiments of the present disclosure.

In some embodiments, a system 200 for attenuation correction includes a first obtaining module 210, a second obtaining module 220, and a reconstruction module 230.

In some embodiments, the first obtaining module 210 is configured to obtain radiological coincidence event data of a target object.

In some embodiments, the second obtaining module 220 is configured to obtain transmission data.

In some embodiments, the reconstruction module 230 is configured to obtain an attenuation-corrected radiological image by performing an attenuation correction and reconstruction based on the transmission data and the radiological coincidence event data. The transmission data includes backscattering coincidence event data of the target object.

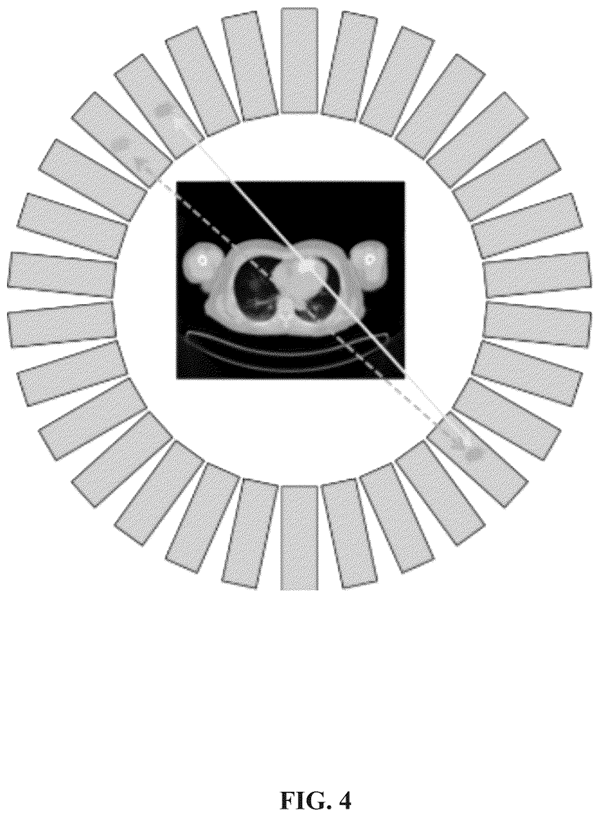

More descriptions about the transmission data, the target object, the radiological coincidence event data, the radiological image, the attenuation correction, and the backscattering coincidence event data may be found in FIG. 3, FIG. 4, and their related descriptions.

It is noted that the above description of the system for attenuation correction and the modules thereof are for descriptive convenience only, and does not limit the present disclosure to the scope of the cited embodiments. It is to be understood that for those skilled in the art, after understanding the principle of the system, it may be possible to arbitrarily combine the individual modules or form a sub-system to be connected to the other modules without departing from this principle. In some embodiments, the first obtaining module 210, the second obtaining module 220, and the reconstruction module 230 in FIG. 2 are different modules in a single system, or a single module realizing the functions of two or more of the above-mentioned modules. For example, the individual modules share a common storage module, or the individual modules each has a respective storage module. Deformations such as these are within the scope of protection of the present disclosure.

FIG. 3 is a flowchart illustrating an exemplary process for attenuation correction according to some embodiments of the present disclosure. As shown in FIG. 3, a process 300 includes one or more of the following operations. In some embodiments, the process 300 is performed by the processing device 120.

In 310, radiological coincidence event data of a target object may be obtained. In some embodiments, operation 310 is performed by the processing device 120 or the first obtaining module 210.

The radiological coincidence event data refers to the data related to the radiation coincidence event. For example, a count, a trajectory, etc., of the radiological coincidence events of the target object. The radiological coincidence events are also known as positive and negative electron annihilation coincidence events. A tracer needs to be introduced into the target subject before PET scanning is performed. The tracer emits positrons during the PET scanning. A great count of negatively charged electrons are naturally present in the target object, the positrons have the same mass and opposite charge as the electrons, and annihilation occurs when the positron collides with the electron (also known as an “annihilation event” or a “coincidence event”). The annihilation produces gamma photons (or radiation rays) of 511 keV energy in two opposite directions. A line connecting two γ-photons may be called a line of response (LOR).

In some embodiments, the processing device 120 obtains the radiological coincidence event data for the target object through the scanning device 110.

In some embodiments, the processing device 120 obtains single event data of the target object; determines whether an energy and an arrival time of the single event data coincide to a second preset rule; and in response to the single event data coincides to the second preset rule, the processing device 120 determines the single event data that coincides with the second preset rule as the radiological coincidence event data. More descriptions about the target object, the single event data, the energy, and the arrival time may be found in the foregoing related descriptions.

The second preset rule may be set based on experience or demands. In some embodiments, the second preset rule includes: if the energies of two different single events in the single event data are both within a third preset energy window, and a difference between the arrival times of the two different single events is within a second preset time window; then data corresponding to the two different single events is the radiological coincidence event data.

The third preset energy window refers to an energy interval used to determine whether two single events are radiological coincidence events.

The second preset time window refers to a preset time interval used to distinguish whether a single event is a radiological coincidence event.

In some embodiments, the third preset energy window and the second preset time window are determined in a variety of manners. For example, the third preset energy window and the second preset time window are preset by experience or demands. Also, for example, the third preset energy window and the second preset time window are set based on one or more scanning device parameters. The scanning device parameter refers to data information related to the scanning device, for example, an axial length, an energy resolution, etc., of the scanning device. The processing device 120 may obtain the third preset energy window and the second preset time window by checking a table. The table includes different scanning device parameters and their corresponding third preset energy windows and second preset time windows. The table may be obtained by preset, historical data, etc.

In some embodiments, the processing device 120 presets parameters (the third preset energy window, the second preset time window, etc.) in the second preset rule, and determine whether the single event data complies with the second preset rule, and if the single event data complies with the second preset rule, the processing device 120 determines the single event data as the radiological coincidence event data.

For example, the processing device 120 sets the third preset energy window to be 425 keV-800 keV and the second preset time window to be 4.4 ns based on a PET scanning device, etc., and selects two different single events in the single event data. A single event 6 occurs before a single event 5; an energy of the single event 5 and an energy of the single event 6 are compared with the third preset energy window, and a difference between arrival times of the single event 5 and the single event 6 is compared with the second preset time window. If the energy of the single event 5 and the energy of the single event 6 are located in the third preset energy window, and the difference between the arrival times of the single event 5 and the single event 6 is located in the second preset time window, then the processing device 120 determines that the data corresponding to the single event 5 and the data corresponding to the single event 6 belongs to the radiological coincidence event data.

In some embodiments, the single event data is collected simultaneously, screened through different preset energy windows and preset time windows, and then the transmission data and the radiological coincidence event data are obtained. In some embodiments, the scanning device 110 uses an extended energy window for collecting the single events. It is understood that the extended energy window has a greater window and is capable of detecting a plurality of types of events (e.g., lutetium background radiation events, object scattering events, inter-crystal scattering events, etc.). Parameters of the extended energy window may be set based on experience or demands, e.g., the parameters of the extended energy window may include a low level discriminator (LLD): 100 keV; a high level discriminator (ULD): 1024 keV.

In some embodiments of the present disclosure, by obtaining the single event data of the target object, and determining the single event data that satisfies the second preset rule as the radiological coincidence event data, the single event data is comprehensively screened from the dimensions of the energy and the arrival time, so as to exclude a noise and interfering signals, and to improve an accuracy and a reliability of the screening of the radiological coincidence event data.

In 320, obtaining transmission data related to the target object may be obtained. In some embodiments, operation 320 is performed by the processing device 120 or the second obtaining module 220.

The transmission data refers to data that transmits the target object. The target object refers to an object to be scanned, for example, a living organism, a mold body, etc. The living organism may be a human body or an animal, etc., and the mold body may be a mold body of a variety of materials and shapes, for example, a water mold, a gel material mold body, a cylinder, a cuboid, etc. In some embodiments, the transmission data includes at least one of the following: backscattering coincidence event data of the target object or lutetium background event data of the imaging device.

The backscattering coincidence event data refers to data related to backscattering coincidence events. For example, a count, a trajectory, etc., of the backscattering coincidence events. A backscattering coincidence event refers to an event where two scattered photons are incorrectly detected by different detectors within a coincidence time window.

In a PET detector, two 511 keV γ-rays interact with matter to produce a photoelectric effect and Compton scattering. The photoelectric effect refers that the γ-rays interact with electrons in the matter and transfers all of their energy to an electron, which breaks away from atom. The Compton scattering refers that the γ-rays interacts with the electrons in the matter, but only a part of the energy is transferred to the electron, and the γ-rays change direction.

In the present disclosure, attention is given to a physical process by which the γ rays undergo the Compton scattering in the PET detector. One or both of the γ-rays undergo the Compton scattering in a first crystal (shown by the dashed line in FIG. 4), with some energy transferred to the electrons, and the γ-rays undergo a backscattering (the backscattering refers to a phenomenon of waves, particles, or signals reflecting back from a direction from which they came), and penetrate back into the target object. Eventually, this γ-ray is detected by another crystal on the other side (as shown by the solid line in FIG. 4). After detection, the backward scattering event forms a transmission LOR for a transmission image estimation.

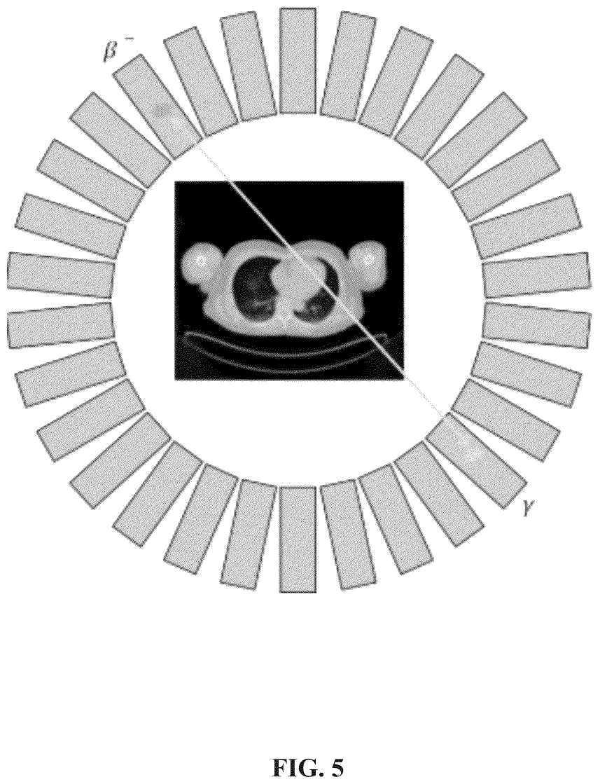

The lutetium background event data refers to the coincidence event data generated by a lutetium spontaneous background radiation of the crystals in a scanning device. For example, a count, a trajectory, etc., of the lutetium background event. As shown in FIG. 5, a common PET system uses a lutetium oxyorthosilicate (LSO) or a (LYSO) crystal as scintillation crystals containing isotope Lu-176, which is capable of generating the spontaneous background radiation.

In some embodiments, the processing device 120 obtains the transmission data through the scanning device 110. For example, the processing device 120 collects a background radiation signal from the scanning device to obtain the lutetium background event data. For another example, the processing device 120 scans the target object by the scanning device 110 to obtain the backscattering coincidence event data.

In some embodiments, the processing device 120 obtains the single event data of the target object; determine whether the energy and the arrival time of the single event data conforms to the first preset rule; and in response to the single event data conforming to the first preset rule, determine that the conforming to the first preset rule, determine that the single event data that meets the first preset rule is transmission data.

The single event data refers to data related to a single event. For example, the single event data includes counts, trajectories, etc., of a plurality of single events. The processing device 120 may obtain the single event data by scanning through the scanning device 110. The arrival time refers to a time when the detector detects the single event. The processing device 120 may scan the energy and the arrival time of the single event data obtained by the scanning device 110.

The first preset rule may be set based on experience or demands. In some embodiments, the first preset rule includes: if two different single events in the single event data have energies that are within a first preset energy window and a second preset energy window, respectively, and a difference between the arrival times of the two different single events is within the first preset time window; then the data corresponding to the two different single events is the transmission data.

Understandably, the energy and the arrival time of the transmission data have certain features, and in a case of the backscattering coincidence event data, a backscattering coincidence event is an event where the signal is reflected back through an object, and the arrival time of the backscattering coincidence event is longer than the arrival time of a directly transmitted signaling event. Therefore, by setting an appropriate arrival time threshold, it is possible to distinguish the directly transmitted signaling events from the backscattering coincidence events. Correspondingly, there is a loss of energy during a reflection process, and the energy of the backscattering coincidence event is typically weaker than the energy of the directly transmitted signaling event. Therefore, the backscattering coincidence event is screened out by setting a suitable energy threshold. Similarly, the lutetium background event data is screened out by setting a suitable energy threshold.

The first preset energy window refers to an energy interval for determining whether the single event data that occurs later is the transmission data. The second preset energy window refers to an energy interval for determining whether the single event data that occurs first is the transmission data. The first preset energy window and the second preset energy window may be set based on experience or demands.

The first preset time window refers to a preset time interval for distinguishing whether the single event data is the transmission data. The first preset time window may be set based on experience or demands.

In some embodiments, the processing device 120 presets parameters in the first preset rule (the first preset energy window, the second preset energy window, the first preset time window, etc.), compares the single event data with the first preset rule, and if the single event data coincides with the first preset rule, it is determined to be the transmission data. Different transmission data (e.g., the backscattering coincidence event data, the lutetium background event data, etc.) may be screened by setting different parameters in the first preset rule (the first preset energy window, the second preset energy window, the first preset time window, etc.).

For example, the processing device 120 sets the first preset energy window to 250 keV-380 keV, the second preset energy window to 140 keV-250 keV, and the first preset time window to a theoretical arrival time±3σ. The theoretical arrival time refers to an arrival time of a single event to the detector, which is obtained based on a ratio of a flying distance of the single event (a distance between an annihilation position of the single event and a detection position) to the light speed, σ indicates a system Gaussian time distribution standard deviation, σ=FWHM/2.355, and FWHM indicates a half-height full-width of a Gaussian function. Two different single events are selected from the single event data, and a single event 2 occurs before a single event 1. The energy of the single event 1 is compared with the first preset energy window, the energy of the single event 2 is compared with the second preset energy window, a difference between the arrival times of the single event 1 and the single event 2 are compared with the first preset time window, and if the energy of the single event 1 is within the first preset energy window, the energy of the single event 2 is within the second preset energy window, and the difference between the arrival times of the single event 1 and the single event 2 is within the first preset time window, then it is determined that the data corresponding to the single event 1 and the single event 2 belongs to the backscattering coincidence event data in the transmission data.

For another example, the processing device 120 sets the first preset energy window to be 0 keV-1000 keV, the second preset energy window to be 100 keV-350 keV, and the first preset time window to be the theoretical arrival time±3σ. Two different single events are selected from the single event data, a single event 4 occurs before a single event 3. The energy of single event 3 is compared with the first preset energy window, the energy of single event 4 is compared with the second preset energy window, and a difference between the arrival time of the single event 3 and the arrival time of the single event 4 is compared with the first preset time window. If the energy of the single event 3 is within the first preset energy window, the energy of the single event 4 is within the second preset energy window, and the difference between the single event 3 and the single event 4 is within the first preset energy window, then it is determined that the data corresponding to the single event 3 and the single event 4 belongs to the lutetium background event data in the transmission data.

In some embodiments, the processing device 120 simultaneously collects the single event data and applies the single event data to the determinations of the backscattering coincidence event data and the lutetium background event data in the transmission data. Alternatively, the processing device 120 separately collects the single event data and applies them to the determination of the backscattering coincidence event data and the lutetium background event data in the transmission data, respectively.

In some embodiments of the present disclosure, by obtaining the single event data of the target object, and determining the single event data that coincides with the first preset rule as the transmission data, the single event data is comprehensively screened from the dimensions of the energy and the arrival time, and the noise and the interference signals are excluded, which improves the accuracy and the reliability of the projection data screening.

In 330, the attenuation-corrected radiological image may be obtained by performing the attenuation correction and reconstruction based on the transmission data and the radiological coincidence event data. In some embodiments, operation 330 is performed by the reconstruction module 230.

The radiological image refers to an image obtained by medical radiology techniques for diagnosing and evaluating diseases. For example, a PET image, etc.

The attenuation correction refers to a correction of errors in a medical image due to photon attenuation. Understandably, in an imaging process of the radiological image, an interaction between the emitted photons and the surrounding tissues can generate the photons. These photons are subjected to different degrees of attenuation as they pass through the body tissues, and these attenuations need to be corrected to obtain more accurate radiological images. The attenuation correction may remove the attenuation effect of the photons from different tissues, so as to obtain more accurate radiological images.

In some embodiments, the processing device 120 obtains the attenuation-corrected radiological image by performing the attenuation correction and reconstruction based on the transmission data and the radiological coincidence event data through a preset manner for attenuation correction. The preset manner for attenuation correction may be set based on experience or needs.

In some embodiments, the processing device 120 uses a first machine learning model to obtain the attenuation-corrected radiological image based on the transmission data and the radiological coincidence event data. In some embodiments, the first machine learning model includes, but is not limited to, a convolutional neural network (CNN) model, a recurrent neural network (RNN), a generative adversarial network (GAN) model, a long short-term memory network (LSTM) model, a transformer model, etc. Specifically, an input to the first machine learning model is the transmission data and the radiological coincidence event data, and an output of the first machine learning model is the attenuation-corrected radiological image. The first machine learning model may reconstruct the attenuation-corrected radiological image based on the transmission data and the radiological coincidence event data.

In some embodiments, the first machine learning model is obtained by training an initial first machine learning model using a plurality of first training samples with first labels. Specifically, the first training samples with the first labels are input to the initial first machine learning model, and parameters of the initial first machine learning model are updated through training to obtain the trained first machine learning model. The first training samples of the first machine learning model include sample transmission data and sample radiological coincidence data, and the first training labels include sample radiological images. The sample transmission data and the sample radiological coincidence event data may be historical data collected by a PET imaging device. The sample radiology images may be gold standard radiology images after attenuation correction based on the sample transmission data and the sample radiology coincidence event data.

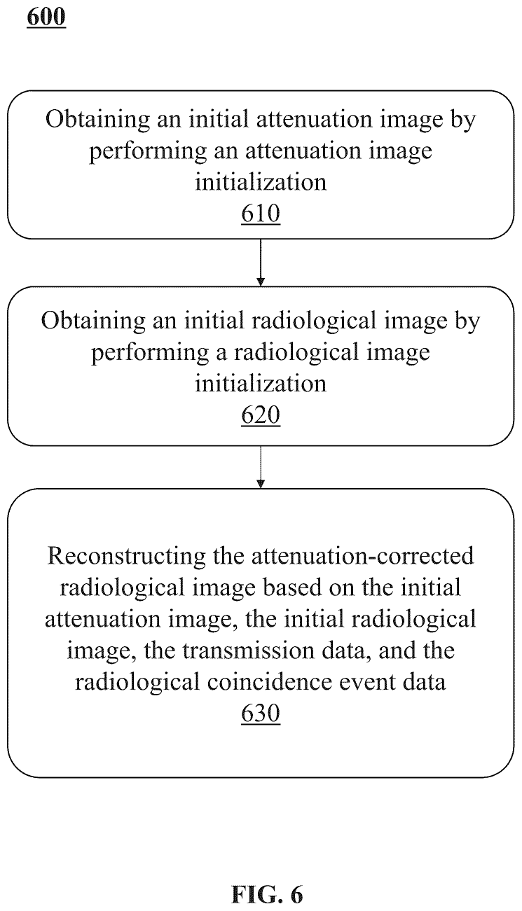

In some embodiments, the processing device 120 performs an iterative reconstruction on the radiological image based on the attenuation image to obtain the attenuation-corrected radiological image. More descriptions of the performing the iterative reconstruction on the radiological image based on the attenuation image to obtain the attenuation-corrected radiological image may be found in FIG. 6 and the related descriptions.

FIG. 6 is a flowchart illustrating an exemplary process for obtaining an attenuation-corrected radiological image of a target object according to some embodiments of the present disclosure. In some embodiments, operation 330 may be performed according to process 600 in FIG. 6. As shown in FIG. 6, an iterative reconstruction process 600 may include one or more of the following operations:

In 610, an initial attenuation image may be obtained by performing an attenuation image initialization.

The attenuation image refers to an image that reflects an attenuation feature of the target object. The initial attenuation image refers to an initially obtained attenuation image.

In some embodiments, the processing device 120 assigns an average value to each pixel value in a preset image (e.g., a mask image), performs the attenuation image initialization, and obtains the initial attenuation image. The average value may be preset based on experience or demands. For example, the processing device 120 assigns an attenuation factor of water to each pixel value in the preset image, performs the attenuation image initialization, and obtains the initial attenuation image.

In some embodiments, the processing device 120 determines a boundary of the target object in the preset image based on the transmission data, and then assigns the average value to each pixel value within the boundary. For example, the processing device 120 determines a plurality of data points associated with the boundary based on the transmission data, and then assigns the attenuation factor of water to each pixel value within the boundary.

In 620, an initial radiological image may be obtained by performing a radiological image initialization.

The initial radiological image refers to an initially obtained radiological image. More descriptions about the radiological image may be found in the preceding related descriptions.

In some embodiments, the processing device 120 assigns the average value to each pixel value in the preset image for the radiological image initialization to obtain the initial radiological image. The average value may be preset based on experience or demands. In some embodiments, the processing device 120 determines the boundary of the target object in the preset image based on radiological coincidence event data, and then assigns the average value to each pixel value within the boundary. More descriptions can be found in the foregoing descriptions with respect to the attenuation image initialization.

In 630, the attenuation-corrected radiological image may be reconstructed based on the initial attenuation image, the initial radiological image, the transmission data, and the radiological coincidence event data.

In some embodiments, the processing device 120 performs the iterative reconstruction using a preset algorithm based on the initial attenuation image, the initial radiological image, the transmission data, and the radiological coincidence event data to obtain the attenuation-corrected radiological image. The preset algorithms may be preset based on experience or demands, for example, the preset algorithms may include a Monte Carlo manner, etc.

The iterative reconstruction refers to a process of multiple rounds of iteration, in which at least a portion of an output of each round is used as a portion of an input of the next round.

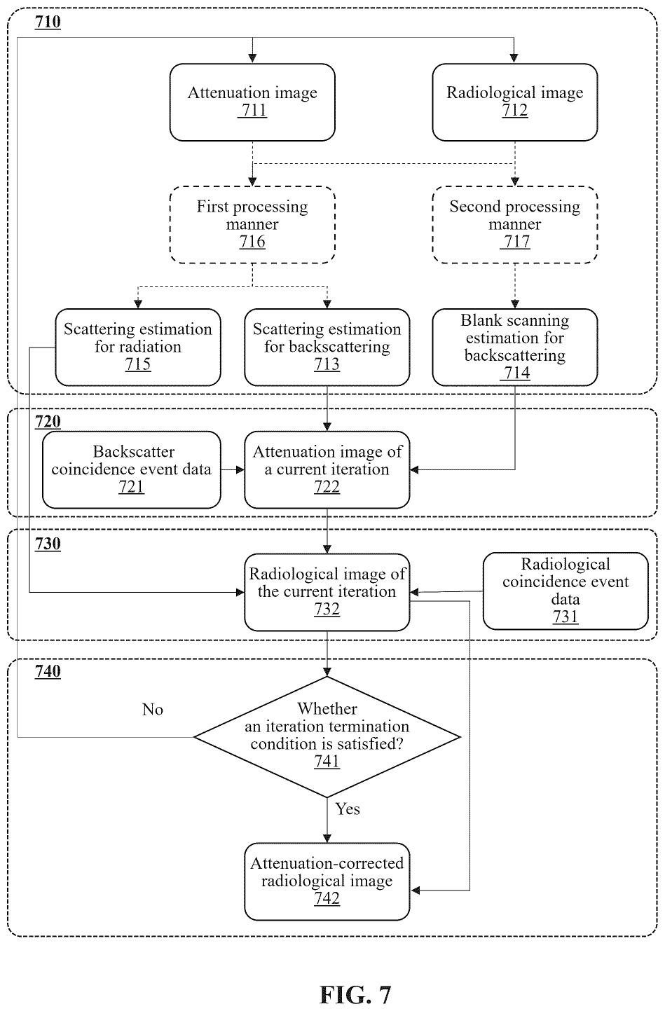

For example, in a process of iteratively reconstructing the attenuation-corrected radiological images, first an initial attenuation image and an initial radiological image are obtained based on an initialization of attenuation images and an initialization of radiological images as iterative inputs for the first round of iteration; in each round of iteration, the iterative input is analyzed and reconstructed to obtain an iterative attenuation image and an iterative radiological image, which are used to update the iterative input of the next iteration. The specific operation of iteration may be found in related contents in FIGS. 7-9.

In some embodiments, the processing device 120 uses a second machine learning model to obtain the attenuation-corrected radiological image based on the initial attenuation image, the initial radiological image, the transmission data, and the radiological coincidence event data. In some embodiments, the second machine learning model includes, but is not limited to, a CNN model, an RNN, a GAN model, an LSTM model, a transformer model, etc. Specifically, the input of the second machine learning model is the initial attenuation image, the initial radiological image, the transmission data, the radiological coincidence event data, and the output of the second machine learning model is the attenuation-corrected radiological image. The second machine learning model may iteratively reconstruct the initial attenuation image and the initial radiological image based on the transmission data and the radiography coincidence event data, and obtain the attenuation-corrected radiological image.

In some embodiments, the second machine learning model is obtained by training an initial second machine learning model using a plurality of second training samples with second labels. Specifically, the second training samples with the second labels are input to the initial second machine learning model, and parameters of the initial second machine learning model are updated through training to obtain the trained second machine learning model. The second training samples of the second machine learning model include sample initial attenuation images, sample initial radiological images, sample transmission data, and sample radiological coincidence event data, and the second training labels include sample radiological images. The sample initial attenuation images and the sample initial radiological images are obtained based on the sample transmission data, the sample radiological coincidence event data. More descriptions of obtaining the sample initial attenuation images and the sample initial radiological images may be found in the relevant descriptions of operation 610 and operation 620. More descriptions of the sample transmission data, the sample radiological coincidence event data, and the sample radiological image may be found in the relevant descriptions of operation 330.

In some embodiments of the present disclosure, by obtaining the transmission data and the radiological coincidence event data and performing the attenuation correction and reconstruction, the attenuation-corrected radiological image is obtained. In this way, the accurate attenuation-corrected radiological image is obtained without the aid of a CT image, thereby avoiding an impact of CT image artifacts and other errors on a correction result, and the process does not increase a complexity of the system, does not increase a scanning time, and does not increase a radiation dose to a patient, while considering a system design and a convenience of user operation, and avoiding an impact on a health of the patient.

FIG. 7 is a schematic diagram illustrating an exemplary process for obtaining an attenuation-corrected radiological image by performing an iterative reconstruction based on backscattering coincidence event data of the target object according to some embodiments of the present disclosure. In some embodiments, operation 630 may be performed according to FIG. 7.

In 710, a scattering estimation for backscattering 713, a blank scanning estimation for backscattering 714, and a scattering estimation for radiation 715 of a target object may be obtained based on an attenuation image, and a radiological image.

The attenuation image of a first iteration may be an initial attenuation image, and the radiological image of the first iteration may be an initial radiological image. The attenuation image of the iterations other than the first iteration may be an attenuation image 711 of the previous iteration, and the radiological image of the iterations other than the first iteration may be a radiological image 712 of the previous iteration.

The scattering estimation for backscattering 713 refers to a distribution estimation of scattering events present in the backscattering coincidence event data. The scattering estimation for radiation 715 refers to a distribution estimation of coincidence events present in the scanning data of the target object. The scanning data refers to data obtained by a scanning device in real time that is used to reconstruct the attenuation-corrected radiological image. For example, a count of all events obtained by scanning the target object, etc. The scattering estimation for backscattering 713 and the scattering estimation for radiation 715 may be expressed in the form of chord diagrams.

In some embodiments, the processing device 120 obtains the scattering estimation for backscattering 713 for and/or the scattering estimation for estimation 715 based on the attenuation image 711 of the previous iteration, the radiological image 712 of the previous iteration, through a preset algorithm. The preset algorithm may be preset based on experience or demands, for example, the preset algorithm may include a Monte Carlo manner, etc.

In some embodiments, the processing device 120 obtains the scattering estimation for backscattering 713 and/or the scattering estimation for radiation 715 based on the attenuation image 711 of the previous iteration and the radiological image 712 of the previous iteration by a first processing manner 716. The first processing manner 716 refers to a processing manner for obtaining the scattering estimation.

In some embodiments, the first processing manner 716 includes one or more of the Monte Carlo manner, a conventional single scatter simulation (SSS) manner, an Energy-based scatter estimation (EBS) manner, etc.

In some embodiments, the first processing manner 716 includes processing the attenuation image of the previous iteration and the radiological image of the previous iteration using a third machine learning model to obtain the scattering estimation for backscattering and the scattering estimation for radiation.

In some embodiments, the third machine learning model includes, but is not limited to, a CNN model, an RNN model, a GAN model, an LSTM model, a transformer model, etc. Specifically, an input to the third machine learning model may be the attenuation image of the previous iteration and the radiological image of the previous iteration, and an output of the third machine learning model may be the scattering estimation for backscattering, and the scattering estimation for radiation. The third machine learning model may analyze the attenuation image of the previous iteration and the radiological image of the previous iteration, and obtain the scattering estimation for backscattering and the scattering estimation for radiation for the current iteration.

In some embodiments, the third machine learning model is obtained by training an initial third machine learning model using a plurality of third training samples with third labels. Specifically, the third training samples with the third labels are input to the initial third machine learning model, and parameters of the initial third machine learning model are updated by training to obtain the trained third machine learning model. The third training samples of the third machine learning model include sample attenuation images, sample radiological images, and the third training labels include sample scattering estimation for backscattering, and sample scattering estimation for radiation. Sample transmission data and sample radiological coincidence event data may be historical data collected by a PET imaging device. The sample scattering estimation for backscattering and the sample scattering estimation for radiation may be determined by analyzing the sample attenuation image and the sample radiological image by other first processing manners.

The blank scanning estimation for backscattering 714 refers to a distribution estimation for backscattering events when there is no target object during backscattering. The blank scanning estimation for backscattering 714 may be expressed in a form of a chord diagram.

In some embodiments, the processing device 120 obtains the blank scanning estimation for backscattering 714 based on the attenuation image 711 of the previous iteration, the radiological image 712 of the previous iteration, by processing using a second processing manner 717.

The second processing manner 717 refers to a processing manner for obtaining a blank scanning estimation.

In some embodiments, the second processing manner 717 includes at least one of a Monte Carlo manner, a checking table manner.

The checking table refers to a data table reflecting a probability distribution of an occurrence of backscattering on a LOR. In some embodiments, the checking table includes a probability distribution of the backscattering of events on each LOR of the PET system being detected by remaining LORs of the PET system. More contents on the LORs may be found in FIG. 3 and the related descriptions.

The checking table may be represented in a matrix form. For example, if there are M LORs, each of which corresponds to an M*1 chordal diagram. The chordal diagram reflects the probability distribution of the backscattering of events on each LOR of the PET system being detected by remaining LORs of the PET system, and the checking table is an M*M matrix, including M count of M*1 chordal diagrams. M is an integer.

Specifically, in some embodiments, the processing device 120 obtains the checking table. In some embodiments, the processing device 120 obtains the checking table through a physical model, the Monte Carlo manner, or other mathematical modeling techniques. For example, the processing device 120 simulates all probabilities of a radiological event incident along a direction of a certain LOR being detected by other LORs by means of the physical model (parameters of the model need to be set autonomously according to an actual scenario), the Monte Carlo manner, etc., and taking the probabilities as a column of data to be filled in the checking table, traverses all the LORs and constructs the checking table.

Further, in some embodiments, the processing device 120 simplifies the checking table based on a symmetry of a positron emission computed tomography system and/or a merging of the LORs.

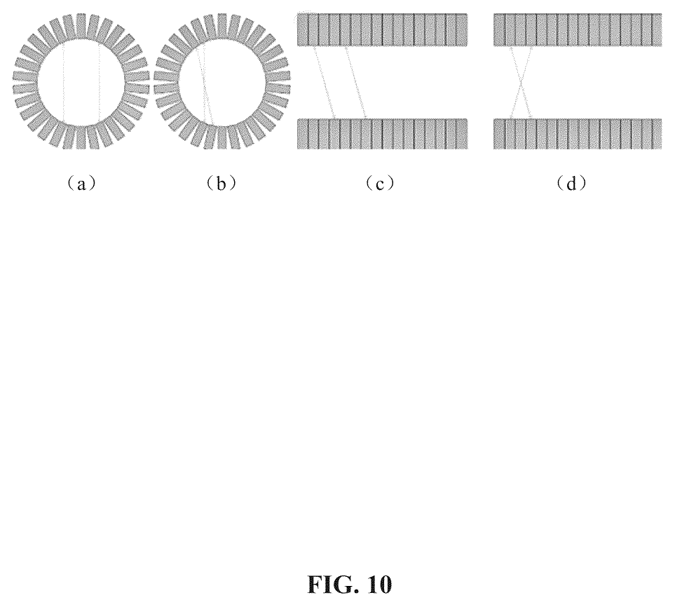

It may be appreciated that the positron emission computed tomography system possesses the symmetry, and the symmetry includes a cross-sectional reflection symmetry of the detector (as shown in (a) in FIG. 10), a cross-sectional rotation symmetry (as shown in (b) in FIG. 10), an axial parallel symmetry (as shown in (c) in FIG. 10), and an axial reflection symmetry (as shown in (d) in FIG. 10). In some embodiments, the processing device 120 reserves, based on the symmetry of the positron emission computed tomography system, among the plurality of LORs where a symmetrical relationship exists, only data of any one LOR (i.e., the data of probability distribution of the backscattering of events on each LOR of the PET system being detected by remaining LORs) in the checking table, and deletes the data of the other LORs, thereby realizing a simplification of the checking table.

In some embodiments, the processing device 120 merges the plurality of LORs into a single LOR based on the merging of LORs, retaining only the data of that LOR in a checking table, realizing a simplification of the checking table. The merging of the LORs may be realized by a merging of detection units of a corresponding detector. For example, the merging of the LORs is realized by merging 4 adjacent detector units, i.e., merging the LORs detected by 4 adjacent detector units into 1. Specific merging rules may be preset based on experience or demands.

Furthermore, in some embodiments, the processing device 120 determines the blank scanning estimation for backscattering 714 based on the simplified checking table, and the scanning data.

In some embodiments, the processing device 120 determines the blank scanning estimation for backscattering 714 based on the simplified checking table and the scanning data by calculation. For example, the processing device 120 calculates a sum of an event count on each LOR in the scanning data, and a product of the probability distribution of the corresponding LOR over the other LORs in the simplified checking table (i.e., the chord diagram of the aforementioned LORs), and determines the sum as the blank scanning estimation for backscattering.

In some embodiments of the present disclosure, by constructing and simplifying the checking table and determining the blank scanning estimation for backscattering 714, the probability distribution of the backscattering of events on the LOR being detected by remaining LORs is obtained by simulation, and thus obtaining an accurate blank scanning estimation for backscattering.

In some embodiments, the second processing manner 717 includes processing the attenuation image of the previous iteration and the radiological image of the previous iteration using a fourth machine learning model, so as to obtain a blank scanning estimation for backscattering.

In some embodiments, the fourth machine learning model includes, but is not limited to, the CNN model, the RNN model, the GAN model, the LSTM model, the transformer model, etc. Specifically, an input to the fourth machine learning model is the attenuation image of the previous iteration and the radiological image of the previous iteration, and an output of the fourth machine learning model is the blank scanning estimation for backscattering. The fourth machine learning model may analyze the attenuation image of the previous iteration and the radiological image of the previous iteration, and obtains the blank scanning estimation for backscattering for a current round of iteration.

In some embodiments, the fourth machine learning model is obtained by training an initial fourth machine learning model using a plurality of fourth training samples with fourth labels. Specifically, the fourth training samples with the fourth labels are input to an initial fourth machine learning model, and parameters of the initial fourth machine learning model are updated through training to obtain the trained fourth machine learning model. The fourth training samples of the fourth machine learning model include sample attenuation images, sample radiological images, and the fourth training labels include sample scattering estimation for backscattering, and sample scattering estimation for radiation. The sample transmission data, the sample radiological coincidence event data may be historical data obtained by the PET imaging device. The blank scanning estimation for backscattering may be determined by analyzing the sample attenuation image and the sample radiological image by other second processing manners.

In some embodiments of the present disclosure, obtaining the scattering estimation for backscattering 713, the scattering estimation for radiation 715, and the blank scanning estimation for backscattering 714 by processing the attenuation image 711 of the previous iteration, the radiological image 712 of the previous iteration through the first processing manner 716 and the second processing manner 717 makes a determination process of the scattering estimation and the blank scanning estimation efficient, accurate, and convenient, which facilitates subsequent updating and reconstruction of the corresponding images.

In 720, an attenuation image of a current iteration 722 may be obtained based on the scattering estimation for backscattering 713, the blank scanning estimation for backscattering 714, and the transmission data. The transmission data may include backscattering coincidence event data 721 of the target object.

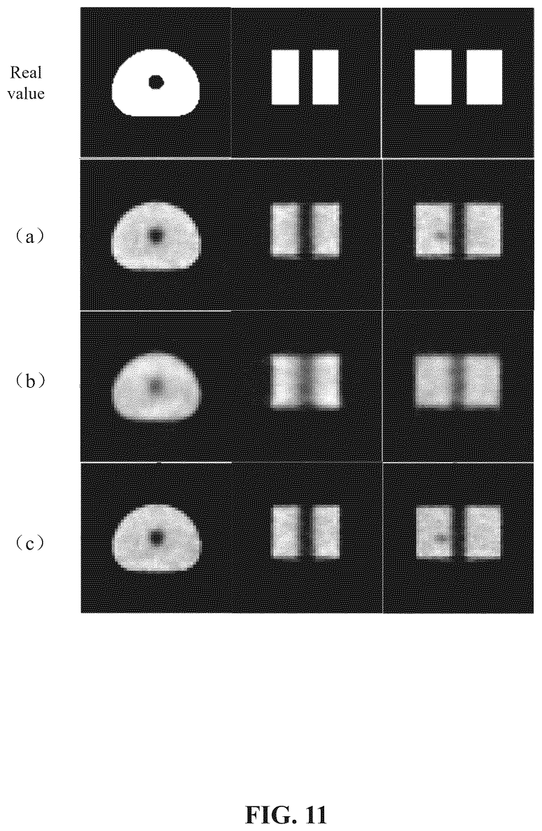

In some embodiments, as shown in (a) in FIG. 11, when iterating the attenuation image based on the backscattering coincidence event data, the attenuation image is iteratively calculated according to Equation (1):

$${μ}_{bs}^{k + 1} = {μ}_{bs}^{k} + \frac{{H}^{T} [ {B}_{bs}^{k} {e}^{- H {μ}_{bs}^{k}} ( 1 - \frac{{y}_{bs}}{{B}_{bs}^{k} {e}^{- H {μ}_{bs}^{k}} + {s}_{bs}^{k} + {r}_{bs}} ) ]}{{H}^{T} [ \frac{{( {B}_{bs}^{k} {e}^{- H {μ}_{bs}^{k}} )}^{2}}{{B}_{bs}^{k} {e}^{- H {μ}_{bs}^{k}} + {s}_{bs}^{k} + {r}_{bs}} H \cdot 1 ]} , \qquad ( 1 )$$

where μ_{bs}^{k+1} denotes the attenuation image of the current iteration, μ_{bs}^{k}, denotes the attenuation image of the previous iteration, B_{bs}^{k} denotes the blank scanning estimation for backscattering, H denotes a system matrix, y_{bs} denotes the backscattering events in the transmission data, s_{bs}^{k} denotes the scattering estimation for backscattering, r_{bs} denotes a chance event estimation for backscattering, the chance event estimation for backscattering referring to a distribution estimation of chance events present in the backscattering coincidence event data, which is obtained by a delay window method (DWM), a singles rate (SR), etc. k denotes a current iteration count.

In 730, a radiological image of the current iteration 732 may be obtained based on the attenuation image of the current iteration 722, the scattering estimation for radiation 715, and the radiological coincidence event data 731.

In some embodiments, the processing device 120 obtains the radiological image of the current iteration 732 based on the attenuation image of the current iteration 722, the scattering estimation for radiation 715, and the radiological coincidence event data 731. In some embodiments, the radiological image is calculated iteratively according to Equation (2):

$${λ}^{k + 1} = \frac{{λ}^{k}}{{( {A}^{k} H )}^{T} \cdot 1} {H}^{T} \frac{{y}_{emission}}{H {λ}^{k} + \frac{{ss}^{k} + rr}{{A}^{k} \cdot 1}} , {A}^{k} = diag ( {e}^{- H {μ}_{*}^{k}} ) , \qquad ( 2 )$$

where λ^{k+1} denotes the radiological image of the current iteration, λ^{k} denotes the radiological image of the previous iteration, yₑₘᵢₛₛᵢₒₙ denotes the radiological coincidence event data, ss denotes a scattering estimation of radiological events, the scattering estimation of radiological events refers to a distribution estimation of scattering events present in the radiological coincidence event data, and rr denotes the chance event estimation for radiological events, which is obtained by the DWW, the SR, etc. μ_{*}^{k} is μ_{bs}^{k} or μₐₗₗ^{k}.

In 740, whether an iteration termination condition 741 is satisfied may be determined. In response to that the iteration termination condition 741 is not satisfied, the next iteration is performed. In response to that the iteration termination condition 741 is satisfied, the radiological image of the current iteration 732 is determined as an attenuation-corrected radiological image 742.

If an iteration situation satisfies a preset iteration condition, the iteration terminates, and the attenuation correction finishes. The preset iteration condition may be that the iteration converges, a preset count of iterations is reached, a difference between the radiological images in the two adjacent iterations is less than a certain threshold, a difference between the attenuation effect chord diagrams in the two adjacent iterations is less than a certain threshold, etc. The attenuation effect chord diagram refers to a forward projection of the attenuation image.

The attenuation-corrected radiological image refers to a radiological image that completes the attenuation correction, which is the radiological image that is output in the last iteration.

In some embodiments of the present disclosure, by obtaining the scattering estimation for backscattering, the blank scanning estimation for backscattering, and the scattering estimation for radiation based on the attenuation image of the previous iteration and the radiological image of the previous iteration; and then obtaining the attenuation image and the radiological image of the current iteration, the iteration is terminated after the iteration termination condition(s) are satisfied, and the attenuation image and radiological image are updated and reconstructed based on real-time data, which makes the attenuation correction process more accurate and efficient.

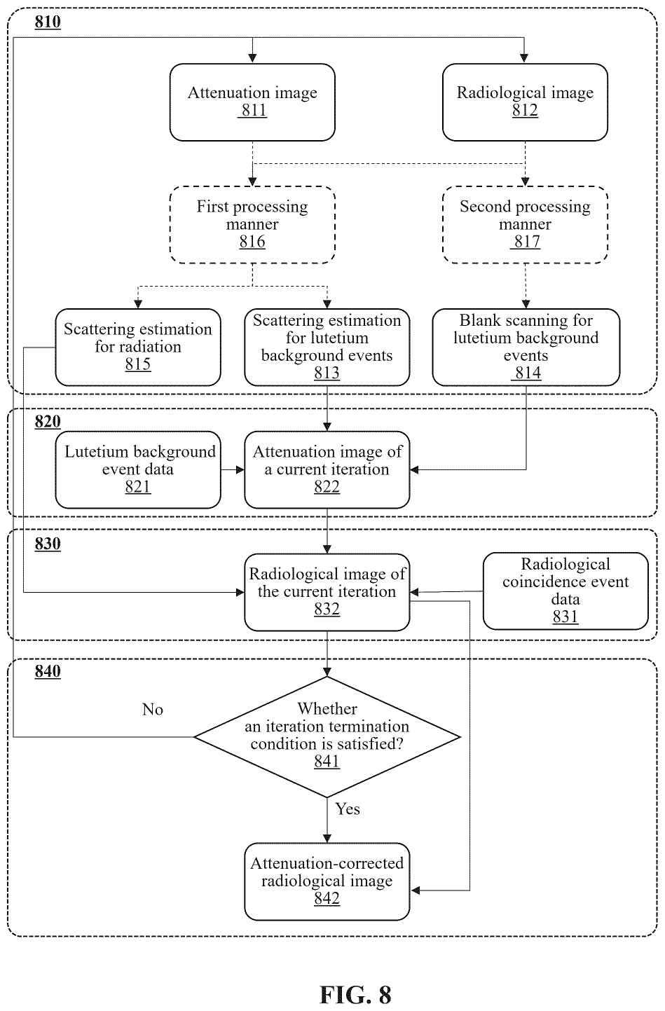

FIG. 8 is a schematic diagram illustrating an exemplary process for obtaining an attenuation-corrected radiological image by performing an iterative reconstruction based on lutetium background event data according to some embodiments of the present disclosure. In some embodiments, operation 630 may be performed according to operations in FIG. 8.

In 810, a scattering estimation for lutetium background events 813, a blank scanning for the lutetium background events 814, and a scattering estimation for radiation 815 of the target object may be obtained based on an attenuation image and a radiological image.

The attenuation image of a first iteration is an initial attenuation image, and the radiological image of the first iteration is an initial radiological image. The attenuation image of the iterations other than the first iteration is an attenuation image 811 of the previous iteration, and the radiological image of the iterations other than the first iteration is the radiological image 812 of the previous iteration.

The scattering estimation for lutetium background events 813 refers to an estimation of a distribution of scattering events present in lutetium background event data. The scattering estimation for radiation 815 refers to a distribution estimation of coincidence events present in scanning data of the target object. The scattering estimation for lutetium background events 813 and the scattering estimation for radiation 815 may be expressed in a form of a chord diagram.

In some embodiments, the processing device 120 obtains, using a preset algorithm, the scattering estimation for the lutetium background events 813 and/or the blank scanning for the lutetium background events 814 based on the attenuation image 811 of the previous iteration and the radiological image 812 of the previous iteration. The preset algorithm is preset based on experience or demands, for example, the preset algorithm includes the Monte Carlo manner, etc.

In some embodiments, the processing device 120 obtains, using a first processing manner, the scattering estimation for the lutetium background events 813 and/or the scattering estimation for radiation 815 based on the attenuation image 811 of the previous iteration and the radiological image 812 of the previous iteration. More descriptions of the first processing manner may be found in the description associated with operation 710.

The blank scanning for the lutetium background events 814 refers to a distribution estimation of the lutetium background events when there are no target objects during a generation of lutetium spontaneous background radiation. In some embodiments, the processing device 120 obtains the blank scanning for the lutetium background events 814 based on the attenuation image 811 of the previous iteration, the radiological image 812 of the previous iteration, by processing using the second processing manner 817. More descriptions of the second processing manner may be found in the description associated with operation 710.

In 820, an attenuation image of a current iteration 822 may be obtained based on the scattering estimation of the lutetium background events 813, the blank scanning for the lutetium background events 814, and the transmission data. The transmission data includes lutetium background event data 821.

In some embodiments, as shown in (b) in FIG. 11, when iterating the attenuation image based on the lutetium background event data, the attenuation image is calculated iteratively according to Equation (3):

$${μ}_{Lu}^{k + 1} = {μ}_{Lu}^{k} + \frac{{H}^{T} [ {B}_{Lu} {{e}^{- H {μ}_{Lu}^{k}}}_{( 1 - \frac{{y}_{Lu}}{{{B}_{Lu} {e}^{- H {μ}_{Lu + {s}_{Lu}^{k} + {r}_{Lu}}^{k}}}^{}} )} ]}{{H}^{T} [ \frac{{( {B}_{Lu} {e}^{- H {μ}_{Lu}^{k}} )}^{2}}{{B}_{Lu} {e}^{- H {μ}_{Lu + {s}_{Lu}^{k} + {r}_{Lu}}^{k}}} H \cdot 1 ]} , \qquad ( 3 )$$

where μ_{Lu}^{k+1} denotes the attenuation image of the current iteration, μ_{Lu}^{k} denotes the attenuation image of the previous iteration, B_{Lu} denotes the blank scanning for the lutetium background events, H denotes the system matrix, y_{Lu} denotes the lutetium background events in the transmission data, sky denotes the scattering estimation for lutetium background events, the scattering estimation for lutetium background events referring to a distribution estimation of the scattered events present in the lutetium background event data, r_{Lu} denotes a chance event estimation for the lutetium background events. The chance event estimation refers to a distribution estimation of chance events present in the lutetium background event data, which is obtained by the DWM, the SR, etc. k denotes the current iteration count. The blank scanning for the lutetium background events is achieved by a blank scanning of the scanning device 110. The blank scanning refers to a direct scanning on air without the target object such as a human body or a molded body, which is regarded as the air.

In 830, a radiological image of a current iteration 832 may be obtained based on the attenuation image of the current iteration 822, the scattering estimation for radiation 815 of the target object, and the radiological coincidence event data 831. More descriptions of obtaining the radiological image of the current iteration based on the attenuation image of the current iteration, the scattering estimation for radiation, and the radiological coincidence event data may be found in the descriptions related to operation 730, which is not repeated herein.

In 840, whether an iteration termination condition is satisfied may be determined. In response to that the iteration termination condition is not satisfied, the process may proceed to a next iteration; or in response to that the iteration termination condition is satisfied, the radiological image of the current iteration 832 may be designated as the attenuation-corrected radiological image. More descriptions of determining whether the iteration termination condition is satisfied may be found in the relevant descriptions of operation 740, which is not repeated here.

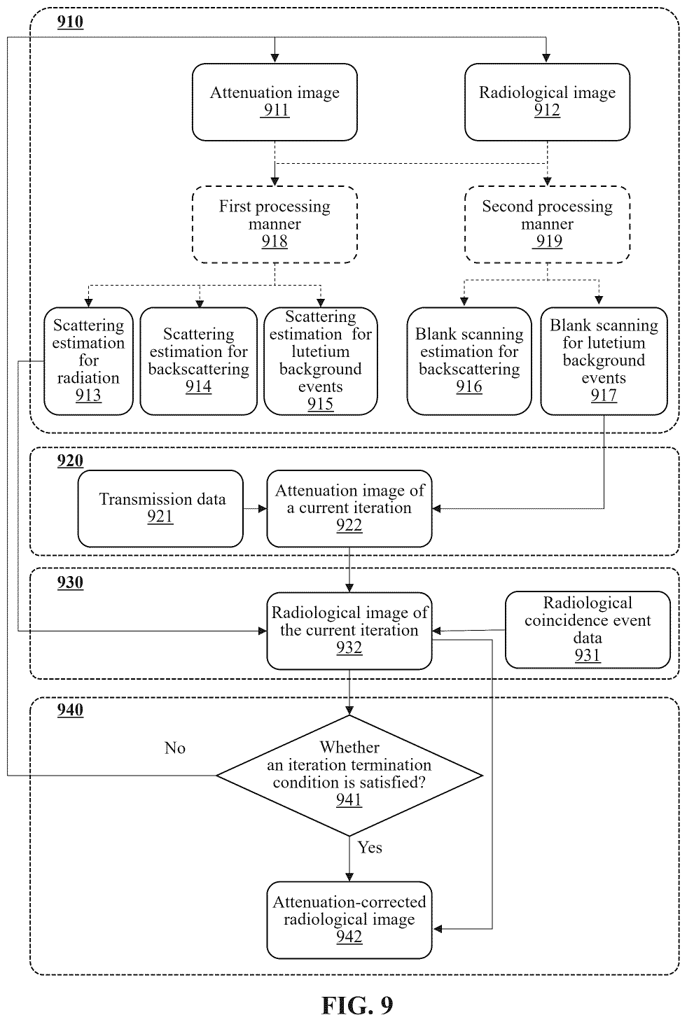

FIG. 9 is a schematic diagram illustrating an exemplary process for obtaining an attenuation-corrected radiological image by performing an iterative reconstruction based on lutetium background event data and backscattering coincidence event data according to some embodiments of the present disclosure. In some embodiments, operation 630 may be performed according to FIG. 9.

In 910, a scattering estimation for backscattering 914, a blank scanning estimation for backscattering 916, a scattering estimation for lutetium background events 915, a blank scanning for lutetium background events 917, and a scattering estimation for radiation 913 of a target object may be obtained based on an attenuation image and a radiological image.

The attenuation image of a first iteration is an initial attenuation image, and the radiological image of the first iteration is an initial radiological image. An attenuation image of the iterations other than the first iteration is an attenuation image 911 of the previous iteration, and a radiological image of the iterations other than the first iteration is a radiological image 912 of the previous iteration.

More descriptions of the scattering estimation for backscattering 914, the blank scanning estimation for backscattering 916, and the scattering estimation for radiation 913 may be found in the related descriptions of operation 710.

More descriptions of the scattering estimation for lutetium background events 915 and the blank scanning for lutetium background events 917 may be found in related descriptions of operation 810.

In 920, an attenuation image of a current iteration 922 may be obtained based on the scattering estimation for backscattering 914, the blank scanning estimation for backscattering 916, the scattering estimation for the lutetium background events 915, the blank scanning for the lutetium background events 917, and the transmission data 921.

In some embodiments, as shown in (c) in FIG. 11, when an attenuation image iteration is performed based on the backscattering coincidence event data and the lutetium background event data together in the transmission data, the attenuation image is calculated iteratively according to Equation (4):

$${μ}_{all}^{k + 1} = {μ}_{all}^{k} + \frac{{H}^{T} [ {e}^{- H {μ}_{all ( 1 - \frac{{y}_{all}}{{B}_{all}^{k} {e}^{- H {μ}_{all + {s}_{all}^{k} + {r}_{all}}^{k}}} )}^{k}} ]}{{H}^{T} [ \frac{{( {B}_{all}^{k} {e}^{- H {μ}_{all}^{k}} )}^{2}}{{B}_{all}^{k} {e}^{- H {μ}_{all + {s}_{all}^{k} + {r}_{all}}^{k}}} H \cdot 1 ]} , \qquad ( 4 )$$

where μₐₗₗ^{k+1} denotes the attenuation image of the current iteration, μₐₗₗ^{k} denotes the attenuation image of the previous iteration, Bₐₗₗ^{k}=B_{bs}^{k}+B_{Lu}, yₐₗₗ=y_{bs}+y_{Lu}, sₐₗₗ^{k}=s_{bs}^{k}+s_{Lu}^{k}, rₐₗₗ=r_{bs}+r_{Lu}.

In 930, a radiological image of the current iteration 932 may be obtained based on the attenuation image of the current iteration 922, the scattering estimation for radiation 913 of the target object, and the radiological coincidence event data 931. More descriptions of obtaining the radiological image of the current iteration based on the attenuation image of the current iteration, the scattering estimation for radiation of the target object, and the radiological coincidence event data may be found in the relevant description of operation 730, which is not repeated here.

In 940, whether an iteration termination condition is satisfied may be determined. In response to that the iteration termination condition is not satisfied, the process may proceed to a next iteration; or in response to that the iteration termination condition is satisfied, the radiological image of the current iteration 932 may be designated as an attenuation-corrected radiological image 942. More descriptions of determining whether the iteration termination condition is satisfied may found in the relevant descriptions of operation 740, which is not repeated here.

One or more embodiments of the present disclosure further provide a computer-readable storage medium storing computer instructions, and when the computer reads the computer instructions in the storage medium, the computer performs the method for attenuation correction as described in any one of the above embodiments.

The basic concepts have been described above, and it is apparent to those skilled in the art that the foregoing detailed disclosure is intended as an example only and does not constitute a limitation of the present disclosure. While not expressly stated herein, those skilled in the art may make various modifications, improvements, and amendments to the present disclosure. Those types of modifications, improvements, and amendments are suggested in the present disclosure, so these types of modifications, improvements, and amendments remain within the spirit and scope of the exemplary embodiments of the present disclosure.

Also, the present disclosure uses specific words to describe embodiments thereof. Such as “an embodiment,” “one embodiment,” and/or “some embodiments” means a feature, structure, or characteristic associated with at least one embodiment of the present disclosure. Accordingly, it should be emphasized and noted that “one embodiment” or “an embodiment” or “an alternative embodiment” in different places in the present disclosure do not necessarily refer to the same embodiment. In addition, certain features, structures, or characteristics in one or more embodiments of the present disclosure may be suitably combined.

Furthermore, unless expressly stated in the claims, an order of the processing elements and sequences described herein, the use of numerical letters, or the use of other names are not intended to qualify the order of the processes and methods of the present disclosure. While some embodiments of the present disclosure that are currently considered useful are discussed in the foregoing disclosure by way of various examples, it should be appreciated that such details serve only illustrative purposes, and that additional claims are not limited to the disclosed embodiments. Rather, the claims are intended to cover all amendments and equivalent combinations that are consistent with the substance and scope of the embodiments of the present disclosure. For example, although the implementation of various components described above are embodied in a hardware device, it may also be implemented as a software only solution, e.g., an installation on an existing server or mobile device.

Similarly, it should be noted that in order to simplify the presentation of the disclosure of the present disclosure, and thereby aid in the understanding of one or more embodiments of the present disclosure, the foregoing descriptions of embodiments of the present disclosure sometimes group multiple features together in a single embodiment, accompanying drawings, or in a description thereof. However, the method of disclosure does not imply that more features are required for the objects of the present disclosure than are mentioned in the claims. Rather, the claimed subject matter may lie in less than all features of a single foregoing disclosed embodiment.

Some embodiments use counts to describe the count of components, attributes, and it should be understood that such counts used in the description of embodiments are modified in some examples by the modifiers “approximately,” “nearly,” or “substantially”. Unless otherwise noted, the terms “approximately,” “nearly,” or “substantially” indicates that a ±20% variation in the stated count is allowed. Correspondingly, in some embodiments, the numerical parameters used in the present disclosure and the claims are approximations, which change depending on the desired characteristics of individual embodiments. In some embodiments, the numerical parameters should consider the specified count of valid digits and use a general digit retention method. While the numerical domains and parameters used to confirm the breadth of their ranges in some embodiments of the present disclosure are approximations, in specific embodiments, such values are set to be as precise as possible within a feasible range.

For each of the patents, patent applications, patent application disclosures, and other materials cited in the present disclosure, such as articles, books, specification sheets, publications, documents, etc., are hereby incorporated by reference in their entirety into the present disclosure. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions and/or use of terms in the present disclosure shall prevail. Finally, it should be understood that the embodiments described herein are only used to illustrate the principles of the embodiments of the present disclosure. Other deformations may also fall within the scope of the present disclosure. As such, alternative configurations of embodiments of the present disclosure are viewed as consistent with the teachings of the present disclosure as an example, not as a limitation. Correspondingly, the embodiments of the present disclosure are not limited to the embodiments expressly presented and described herein.

What is claimed is:

1. A method implemented on a computing device having one or more processors and one or more storage devices for attenuation correction, the method comprising:
obtaining radiological coincidence event data of a target object using an imaging device;
obtaining transmission data related to the target object; and
obtaining an attenuation-corrected radiological image by performing an attenuation correction and reconstruction based on the transmission data and the radiological coincidence event data.

2. The method of claim 1, wherein the transmission data includes at least one of: backscattering coincidence event data of the target object or lutetium background event data of the imaging device.

3. The method of claim 1, wherein the obtaining transmission data includes:
obtaining single event data of the target object;
determining whether an energy and an arrival time of the single event data complies with a first preset rule; and
in response to that the single event data complies with the first preset rule, designating the single event data that complies with the first preset rule as the transmission data.

4. The method of claim 1, wherein the obtaining radiological coincidence event data of a target object includes:
obtaining single event data of the target object;
determining whether an energy and an arrival time of the single event data complies with a second preset rule; and
in response to that the single event data complies with the second preset rule, designating the single event data that complies with the second preset rule as the radiological coincidence event data.

5. The method of claim 1, wherein the obtaining an attenuation-corrected radiological image includes:
obtaining an initial attenuation image by performing an attenuation image initialization;
obtaining an initial radiological image by performing a radiological image initialization; and
reconstructing the attenuation-corrected radiological image based on the initial attenuation image, the initial radiological image, the transmission data, and the radiological coincidence event data.

6. The method of claim 5, wherein the transmission data includes backscatter coincidence event data of backscattering of the target object, and the reconstructing the attenuation-corrected radiological image includes:
obtaining a scattering estimation for backscattering, a blank scanning estimation for backscattering, and a scattering estimation for radiation of the target object based on an attenuation image of a previous iteration, and a radiological image of the previous iteration, wherein the initial attenuation image is used as an attenuation image of an initial iteration, and the initial radiological image is used as a radiological image of the initial iteration;
obtaining an attenuation image of a current iteration based on the scattering estimation for backscattering, the blank scanning estimation for backscattering, and the transmission data;
obtaining a radiological image of the current iteration based on the attenuation image of the current iteration, the scattering estimation for radiation, and the radiological coincidence event data;
determining whether an iteration termination condition is satisfied; and
in response to that the iteration termination condition is not satisfied, proceeding to a next iteration; or in response to that the iteration termination condition is satisfied, designating the radiological image of the current iteration as the attenuation-corrected radiological image.

7. The method of claim 6, wherein the obtaining a scattering estimation for backscattering, a blank scanning estimation for backscattering, and a scattering estimation for radiation of the target object based on an attenuation image of a previous iteration, and a radiological image of the previous iteration includes:
obtaining the scattering estimation for backscattering and the scattering estimation for radiation of the target object by processing in a first processing manner based on an attenuation image of the previous iteration and a radiological image of the previous iteration; and
obtaining the blank scanning estimation for backscattering by processing in a second processing manner based on the attenuation image of the previous iteration and the radiological image of the previous iteration, wherein the second processing manner includes at least one of Monte Carlo algorithm or a table checking operation.

8. The method of claim 7, wherein the imaging device includes a positron emission tomography (PET) system, the backscattering coincidence event data is obtained by the PET system, and a checking table used in the table checking operation includes a probability distribution of the backscattering of events on each line of response (LOR) of the PET system being detected by remaining LORs of the PET system, the table checking operation includes:
obtaining the checking table;
simplifying the checking table based on a symmetry of the PET system and/or a merging of LORs of the PET system; and
determining the blank scanning estimation for backscattering based on the simplified checking table and scan data obtained by scanning the target object.

9. The method of claim 5, wherein the transmission data includes lutetium background event data of the imaging device, and the reconstructing the attenuation-corrected radiological image includes:
obtaining a scattering estimation for lutetium background events of the imaging device, a blank scanning for the lutetium background events, and a scattering estimation for radiation of the target object based on an attenuation image of a previous iteration, and a radiological image of the previous iteration, wherein the initial attenuation image is used as an attenuation image of an initial iteration, and the initial radiological image is used as a radiological image of the initial iteration;
obtaining an attenuation image of a current iteration based on the scattering estimation of the lutetium background events, the blank scanning for the lutetium background events, and the transmission data;
obtaining a radiological image of a current iteration based on the attenuation image of the current iteration, the scattering estimation for radiation of the target object, and the radiological coincidence event data;
determining whether an iteration termination condition is satisfied; and
in response to that the iteration termination condition is not satisfied, proceeding to a next iteration; or in response to that the iteration termination condition is satisfied, designating the radiological image of the current iteration as the attenuation-corrected radiological image.

10. The method of claim 5, wherein the transmission data includes backscattering coincidence event data of the target object and lutetium background event data of the imaging device, and the reconstructing the attenuation-corrected radiological image includes:
obtaining a scattering estimation for backscattering, a blank scanning estimation for backscattering, a scattering estimation for lutetium background events of the imaging device, a scattering estimation for radiation of the target object, based on an attenuation image of a previous iteration, and a radiological image of the previous iteration, wherein the initial attenuation image is used as an attenuation image of an initial iteration, and the initial radiological image is used as a radiological image of the initial iteration;
obtaining an attenuation image of the current iteration based on the scattering estimation for backscattering, the blank scanning estimation for backscattering, the scattering estimation for the lutetium background events, blank scanning data for the lutetium background events, and the transmission data;
obtaining a radiological image of the current iteration based on the attenuation image of the current iteration, the scattering estimation for radiation of the target object, and the radiological coincidence event data;
determining whether an iteration termination condition is satisfied; and
in response to that the iteration termination condition is not satisfied, proceeding to a next iteration; or in response to that the iteration termination condition is satisfied, designating the radiological image of the current iteration as the attenuation-corrected radiological image.

11. The method of claim 1, wherein the obtaining an attenuation-corrected radiological image includes:
obtaining the attenuation-corrected radiological image based on the transmission data and the radiological coincidence event data using a first machine learning model.

12. The method of claim 5, wherein the reconstructing the attenuation-corrected radiological image includes:
obtaining the attenuation-corrected radiological image based on the initial attenuation image, the initial radiological image, the transmission data, and the radiological coincidence event data using a second machine learning model.

13. The method of claim 7, wherein the first processing manner includes processing the attenuation image of the previous iteration, and the radiological image of the previous iteration using a third machine learning model to obtain the scattering estimation for backscattering and the scattering estimation for radiation of the target object.

14. The method of claim 7, wherein the second processing manner includes processing the attenuation image of the previous iteration and the radiological image of the previous iteration using a fourth machine learning model to obtain the blank scanning estimation for the backscattering.

15. A system for attenuation correction, comprising:
at least one storage device including a set of instructions or programs; and
at least one processor configured to communicate with the at least one storage device, wherein when executing the set of instructions or programs, the at least one processor is configured to cause the system to perform operations including:
obtaining radiological coincidence event data of radiation of a target object using an imaging device;
obtaining transmission data related to the target object; and
obtaining an attenuation-corrected radiological image by performing an attenuation correction and reconstruction based on the transmission data and the radiological coincidence event data.

16. The system of claim 15, wherein the obtaining transmission data includes:
obtaining single event data of the target object;
determining whether an energy and an arrival time of the single event data complies with a first preset rule; and
in response to that the single event data complies with the first preset rule, designating the single event data that complies with the first preset rule as the transmission data.

17. The system of claim 15, wherein the obtaining radiological coincidence event data of radiation of a target object includes:
obtaining single event data of the target object;
determining whether an energy and an arrival time of the single event data complies with a second preset rule; and
in response to that the single event data complies with the second preset rule, designating the single event data that complies with the second preset rule as the radiological coincidence event data.

18. The system of claim 15, wherein the obtaining an attenuation-corrected radiological image includes:
obtaining an initial attenuation image by performing an attenuation image initialization;
obtaining an initial radiological image by performing a radiological image initialization; and
reconstructing the attenuation-corrected radiological image based on the initial attenuation image, the initial radiological image, the transmission data, and the radiological coincidence event data.

19. The system of claim 18, wherein the transmission data includes backscattering coincidence event data of the target object and lutetium background event data of the imaging device, and the reconstructing the attenuation-corrected radiological image includes:
obtaining a scattering estimation for backscattering, a blank scanning estimation for backscattering, a scattering estimation for lutetium background events of the imaging device, a scattering estimation for radiation of the target object, based on an attenuation image of a previous iteration, and a radiological image of the previous iteration, wherein the initial attenuation image is used as an attenuation image of an initial iteration, and the initial radiological image is used as a radiological image of the initial iteration;
obtaining an attenuation image of the current iteration based on the scattering estimation for backscattering, the blank scanning estimation for backscattering, the scattering estimation for the lutetium background events, blank scanning data for the lutetium background events, and the transmission data;
obtaining a radiological image of the current iteration based on the attenuation image of the current iteration, the scattering estimation for radiation of the target object, and the radiological coincidence event data;
determining whether an iteration termination condition is satisfied; and
in response to that the iteration termination condition is not satisfied, proceeding to a next iteration; or in response to that the iteration termination condition is satisfied, designating the radiological image of the current iteration as the attenuation-corrected radiological image.

20. A non-transitory computer readable medium storing instructions, the instructions, when executed by at least one processor, causing the at least one processor to implement a method comprising:
obtaining radiological coincidence event data of radiation of a target object using an imaging device;
obtaining transmission data related to the target object; and
obtaining an attenuation-corrected radiological image by performing an attenuation correction and reconstruction based on the transmission data and the radiological coincidence event data.
